(19) United States
(12) Patent Application Publication
(10) Pub. No.: US 2007/0128140 A1
(43) Pub. Date: Jun. 7, 2007

(54) COMPOSITION INCORPORATING EMOLLIENT OILS INTO BAR SOAP PRODUCTS

(76) Inventor: E. Gary Myers, Scottsdale, AZ (US)

Correspondence Address: SNELL & WILMER, LLP (Dial Corp), ONE ARIZONA CENTER, 400 E. VAN BUREN, PHOENIZ, AZ, 85004-2202, US

(21) Appl. No.: 11/560,980

(22) Filed: Nov. 17, 2006

Related U.S. Application Data
(60) Provisional application No. 60/597,256, filed on Nov. 18, 2005.

(51) Int. Cl. A61K 8/37; A61K 8/18

(52) U.S. Cl. 424/70.1; 510/141

(57) ABSTRACT

A composition for incorporating emollient oils into soap, soap/synthetic combinations, or all synthetic soap products is provided. The composition comprises a dispersion, soap pellets and additional ingredients.

### CROSS-REFERENCE TO RELATED APPLICATIONS

This application claims priority to U.S. Provisional Patent Application Ser. No. 60/597,256, entitled “Composition Incorporating Emollient Oils Into Soap Products,” filed Nov. 18, 2005.

### FIELD OF INVENTION

The present invention is generally directed towards a bar soap product composition. More particularly, the present invention is directed towards a bar soap product composition incorporating high levels of emollient oils.

### BACKGROUND OF THE INVENTION

Personal cleansing products have attempted to meet a variety of product characteristics desired by consumers. In general, a suitable cleansing product must exhibit good cleaning and lathering characteristics while still being mild to the skin. More beneficial are products which do not irritate the skin and leave the skin feeling moisturized.

One type of traditional moisturizing formulation includes oil and water emulsions. These formulations are created by emulsifying non-soluble skin conditioning oils into water based cleansing formulations. These formulations are balanced between the cleansing properties of the water phase and the softening effects of the oils deposited on the skin. Stability of these formulations is achieved by using an excess of surfactants present in the emulsion phase. However, emulsification of oils in water based cleansers negatively impacts the lathering and cleansing properties of the cleansers. High oil content often drastically reduces lathering ability of the product. Also, high oil content added to soap during production may leave the mass sticky and slippery making processing of soap bars difficult and inefficient. Furthermore, surfactants, responsible for the cleansing effects, are often irritating to the skin. As such, increasing surfactant use so that more emollient oil can be incorporated into a particular composition may have no net benefit to the softening and/or cleansing characteristics of the soap.

Thus, there is a need to stably increase the amount of emollient oil in soap products without significantly impacting lather, cleansing properties, and processability while at the same time limiting skin irritation and increasing skin conditioning effects.

### SUMMARY OF THE INVENTION

While the way in which the present invention address the disadvantages of the prior art will be discussed in greater detail below, in general, the present invention provides a soap product composition that effectively cleanses and softens the skin. Additionally, the present invention provides a method for incorporating high levels of emollient oil into soap product compositions thereby increasing skin conditioning effects without destroying processability.

In accordance with an exemplary embodiment of the present invention, the bar soap product composition preferably comprises a dispersion (e.g., comprised of an emollient oil, a surfactant, and water), soap, and optional additional ingredients, for example, preservatives, fragrances, color adjusters, antibacterial agents, and/or vitamins. In an exemplary embodiment, the highly stable dispersion is present in an amount preferably from about 3 to about 12 percent by weight of the bar soap product composition.

### DETAILED DESCRIPTION

The following descriptions are of exemplary embodiments of the invention only, and are not intended to limit the scope or applicability of the invention in any way. Rather, the following description is intended to provide convenient illustrations for implementing various embodiments of the invention. As will become apparent, various changes may be made in the compositions described in these embodiments without departing from the spirit and scope of the invention.

In accordance with various aspects of the present invention, a soap product composition that effectively cleanses and softens the skin is provided. For example, in accordance with various embodiments of the invention, the soap product may be formulated so that high levels of emollient oils are delivered to the skin by vigorously rubbing the soap product on the skin under running water. As a result, the oils are easily felt on the body parts during washing without a decrease in lather volume. Upon towel drying, the oils leave the body parts washed feeling soft and treated. While the invention will be described in this context, it should be appreciated that other uses as are now known or hereafter devised by those skilled in the art may be made of the compositions set forth herein.

In accordance with various embodiments of the present invention, the soap product composition preferably comprises a highly stable dispersion, soap, and optional additional ingredients, for example, preservatives, fragrances, color adjusters, antibacterial agents, and/or vitamins.

In accordance with one aspect of one embodiment of the present invention, the soap product composition comprises a highly stable dispersion. As mentioned above, one type of traditional moisturizing formulation includes emulsions of oil and soap. Stability of these formulations is achieved by using an excess of surfactants present in the emulsion phase. A dispersion is a generally stable or unstable mixture of at least two immiscible substances. Providing a dispersion of emollient oil that is stable prior to mixture with soap pellets, for example, significantly decreases the amount of surfactants necessary to stabilize the soap product composition. By so doing, this invention provides for an increased amount of emollient oil that does not noticeably impact lather, cleansing properties, or processability, increase skin irritation, or decrease skin conditioning effects.

In general, the dispersion may be comprised of other composition ingredients during processing to deliver an effective amount of oil into the soap product composition. Exemplary compositions may include: one or more emollients, one or more humectants, one or more preservatives, one or more antibacterial agents, one or more fragrances, one or more surfactants, whether it be anionic, cationic, nonionic, ampholytic, amphoteric, zwitterionic surfactants, or the like, one or more antioxidants, one or more colorants, and one or more neutralizers.

The highly stable dispersion enables greater amounts of emollient oil to be incorporated into the desired soap product composition than without use of the highly stable dispersion. Without being limited to any theory, it is believed the oil becomes stabilized in the dispersion phase, and initially separated from the base waxy soap, but thereafter dispersed thoroughly therethrough, thereby increasing composition stability. Because of this prior stabilization in the dispersion phase and initial separation, there is increased stability in the overall soap product composition, and less surfactant or other stabilizer is needed. Only a small amount of surfactant is needed to maintain the stable dispersion which delivers the oil to the skin while maintaining lather from the base soap as it solubilizes in water as the bar is rubbed. This in turn reduces the irritant effect that increased surfactant levels may cause.

That being said, the highly stable dispersion is preferably strong enough to survive throughout product processing but should be able to be activated by rubbing the product between the hands or other skin surfaces in the presence of water. Upon activation, the emollient oils are delivered to the skin leaving the skin feeling soft and conditioned.

As such, any composition capable of stably delivering oil into a soap product composition may be suitable as a highly stable dispersion. For example, in accordance with various embodiments of this aspect of the present invention, the highly stable dispersion comprises an emollient oil, a surfactant, and water.

In an exemplary embodiment, the highly stable dispersion is present in an amount from about 3 to about 12 percent by weight of the soap product composition. More preferably, the dispersion is present in an amount from about 5 to about 10 percent, and most preferably from about 6 to about 9 percent by weight of the soap product composition.

In accordance with one embodiment of the present invention, the highly stable dispersion comprises one or more emollient oils. Emollient oils are known for moisturizing and softening the skin by depositing a layer of oil on the skin to slow water loss and increase water content. As such, any emollient may be suitable for use in the highly stable dispersion. Exemplary emollients within the spirit and scope of the invention may include, but are not limited to algae extract, borage seed oil, carrageenan extract, castor oil, corn oil, evening primrose oil, grape seed oil, jojoba oil, kukui nut oil, lecithin, macadamia oil, oat kernel meal, pea extract, pecan oil, safflower oil, sesame oil, shea butter, soybean oil, sunflower oil and/or sweet almond oil.

In accordance with various exemplary embodiments, the emollient oils are present in an amount from about 75 to about 95 percent by weight of the dispersion. More preferably, the oils are present in about 85 to about 93 percent by weight, and most preferably, the oils are present in about 88 to about 90 percent by weight of the dispersion. In accordance with these exemplary embodiments, the emollient oils comprise a combination of soybean and safflower oil present in a 50%/50% mixture. However, any single oil, combination of oils or type of emollient now known or hereafter devised is suitable.

In accordance with an exemplary embodiment of the present invention, the dispersion comprises an effective amount of one or more surfactants. Surfactants such as anionic, cationic, nonionic, ampholytic, amphoteric, or zwitterionic surfactants, and/or mixtures thereof capable of stably dispersing oil in oil-in-water dispersions are suitable.

Specific surfactants that can be used in the dispersion include, but are not limited to, lauryl sulfates, octyl sulfates, 2-ethylhexyl sulfates, lauramine oxide, decyl sulfates, tridecyl sulfates, cocoates, lauryl sarcosinates, lauryl sulfosuccinates, linear C.sub.10 diphenyl oxide disulfonates, lauryl sulfosuccinates, lauryl ether sulfates (1 and 2 moles ethylene oxide), myristyl sulfates, oleates, stearates, tallates, cocamine oxide, decylamine oxide, myristamine oxide, ricinoleates, cetyl sulfates, and similar surfactants. Suitable anionic surfactants include, but are not limited to, compounds in the classes known alkyl sulfates, alkyl ether sulfates, alkyl ether sulfonates, sulfate esters of an alkylphenoxy polyoxyethylene ethanol, alpha-olefin sulfonates, beta-alkoxy alkane sulfonates, alkylaryl sulfonates, alkyl monoglyceride sulfates, alkyl monoglyceride sulfonates, alkyl carbonates, alkyl ether carboxylates, fatty acids, sulfosuccinates, sarcosinates, octoxynol or nonoxynol phosphates, taurates, fatty taurides, fatty acid amide polyoxylethelyne sulfates, isethionates, or mixtures thereof.

Preferably, the surfactant is mild. That is, the surfactant provides enough cleansing benefit while not overly irritating the skin. Furthermore, an effective amount of surfactant is that which is capable of forming a highly stable dispersion of emollient oil droplets, but is activated upon rubbing with the skin in the presence of water. In a preferred embodiment, the surfactant in the dispersion is sodium laureth sulfate present in an amount from about 0.5 to about 5 percent by weight of the dispersion. More preferably, the sodium laureth sulfate surfactant is present in an amount from about 0.8 to about 2.5 percent, and most preferably, in an amount from about 1.0 to about 1.5 percent by weight of the dispersion.

In accordance with this aspect of the present invention, the highly stable dispersion further comprises water. In an exemplary embodiment, the water is present in an amount from about 5 to about 15 percent by weight of the dispersion. More preferably, the water is present from about 8 to about 12 percent, and optimally, the water is present in about 9 to about 10 percent by weight of the dispersion.

In accordance with another aspect of the present invention, the soap product composition comprises soap. As used herein, the term “soap” is defined as any water-soluble salt of those fatty acids that contain about 8 or more carbon atoms. In an exemplary embodiment of the present invention, the soap mixture comprises at least one of sodium tallowate, sodium cocoate, sodium palmitate, sodium stearate and sodium palm kernelate, salts of fatty acids having alkyl chain lengths with 8 to 18 carbons. In a preferred embodiment of the invention, the soap mixture suitably comprises a blend of sodium cocoate, sodium palmate, sodium tallowate, sodium palmitate, sodium stearate and sodium palm kernelate. It will be appreciated, however, that other fats and oils from which soaps may be produced can be employed, such as babassu oil, soybean oil, cottonseed oil, rapeseed oil or other comparable vegetable product, whale or fish oils and lards, and the like. Various other animal fats and oils may also be employed to produce soaps similar to tallowate/cocoate/palmitate/stearate/palm kernelate soaps mentioned above. Generally, fatty acids with alkyl chains having 10 to 18 carbons are most desirable for soap production, as shorter alkyl chains may not produce the desired soap properties. Fatty acids with chain lengths having 20 carbons or more have very little solubility and may impede lathering.

In various embodiments, the soap may comprise traditional soap, soap/synthetic combinations, or all synthetic formulations. In accordance with an exemplary embodiment, the soap product is in the form of pellets or granulars suitable for manufacturing into soap bars. In accordance with this exemplary embodiment, the soap pellets or granulars are present in an amount from about 88 to about 97 percent by weight of the soap product composition. More preferably, the soap pellets or granulars are present in an amount from about 90 to about 95 percent, and most preferably, from about 93 to about 95 percent by weight of the soap product composition.

In various embodiments of the present invention, the soap product composition may optionally be configured for a variety of particular end-use purposes. Any additional ingredients may be added to the soap product composition; however, these optional ingredients should not interfere with the cleaning efficacy or the dispersion properties of the composition. For example, soap product compositions in accordance with the present invention may comprise one or more of a naturalizer, a preservative, a fragrance, a color adjuster, an antibacterial agent, and/or a vitamin, such as Vitamin E and/or Vitamin A. In accordance with various embodiments, a color slurry may be added to the soap product composition which is a dye or pigment dispersed in water or some other solvent and may contain TiO₂ and a small amount of detergent to help stabilize the dispersion. Furthermore, in accordance with these embodiments, the slurry may also contain an antibacterial agent such as a bisguanidine (e.g., chlorhexidine digluconate), diphenyl compounds, benzyl alcohols, trihalocarbanilides, quaternary ammonium compounds, ethoxylated phenols, and phenolic compounds, such as halo-substituted phenolic compounds, like PCMX (i.e., p-chloro-m-xylenol) and Triclosan (i.e., 2,4,4′-tri-chloro-2′hydroxy-diphenylether), Triclocarban, Triclocarbanilide, or other now known or hereafter devised germicides.

Table 1 shows 2 exemplary bar soap product compositions within the spirit and scope of the invention.

TABLE 1

| | | Exemplary Bar | Exemplary Bar |
|---|---|---|---|
| | | Soap Product | Soap Product |
| | | Composition | Composition |
| | | #1 | #2 |
| | | Weight percent | |
| Dispersion | 89.0% oil (50% | 5.0 | 7.2 |
| | soybean oil/50% | | |
| | safflower oil) | | |
| | 9.8% water | | |
| | 1.2% surfactant | | |
| Soap Pellets | | 95.0 | 92.8 |
| Total | | 100 | 100 |

| | | |
|---|---|---|
| Oil Content in Final Bar Soap Product | 4.5 | 6.41 |

In the examples described above, the soap pellets and the oil/water/surfactant dispersion were blended using known soap plodding devices. As such, the soap pellets and the dispersion were blended in an intensive mixer, refined through a twin screw plodder with screens, and through a duplex vacuum plodder prior to being cut into soap slugs that were then stamped on a soap press into bars. The formula produced unexpected results, since the formula was easily made into soap bars with good efficiency on a soap line. Typically this load of oils and other liquids would make the soap mass extremely sticky and slippery, cutting down the ability to move the soap down the soap finishing line, and produce sticky bars that are soft and extremely difficult to stamp, making for a low efficiency process with high bar defects from the soft soap. A second unexpected result was related to bar performance. As noted above, normally, high oil loads drastically reduce lather. However, the test bars lathered very well indicating that the soap and oil in the dispersion were effectively kept separated during the soap processing, and the oils were activated by the water and mechanical action of hand washing. The presence of the oil was immediately noticeable during hand washing, and the hands felt soft and treated after towel drying.

Finally, it should be understood that various principles of the invention have been described in illustrative embodiments. However, many combinations and modifications of the above-described formulation, proportions, elements, materials and components, used in the practice of the invention, in addition to those not specifically described, may be varied and particularly adapted to specific environments and operating requirements without departing from those principles. Other variations and modifications of the present invention will be apparent to those of ordinary skill in the art, and it is the intent that such variations and modifications be covered.

What is claimed is:

1. A bar soap product composition comprising:
a stable dispersion from about 3 to about 12 percent by weight of the soap product composition comprising:
at least one emollient oil from about 75 to about 95 percent by weight of the dispersion;
at least one surfactant from about 0.5 to about 5 percent by weight of the dispersion; and
water from about 5 to about 15 percent by weight of the dispersion;
soap pellets from about 88 to about 97 percent by weight of the soap product composition; and
optional additional ingredients comprising at least one of: a preservative, a fragrance, a color adjuster, an antibacterial agent, and a vitamin.

2. The composition of claim 1, wherein the stable dispersion is present from about 5 to about 10 percent by weight of the soap product composition.

3. The composition of claim 2, wherein the stable dispersion is present from about 6 to about 9 percent by weight of the soap product composition.

4. The composition of claim 1, wherein the emollient oil is present from about 85 to about 93 percent by weight of the dispersion.

5. The composition of claim 4, wherein the emollient oil is present from about 88 to about 90 percent by weight of the dispersion.

6. The composition of claim 1, wherein the emollient oil comprises a combination of soybean and safflower oil present in equal amounts.

7. The composition of claim 1, wherein the surfactant is present from about 0.8 to about 2.5 percent by weight of the dispersion.

8. The composition of claim 7, wherein the surfactant is present from about 1.0 to about 1.5 percent by weight of the dispersion.

9. The composition of claim 1, wherein the surfactant is sodium laureth sulfate.

10. The composition of claim 1, wherein water is present from about 8 to about 12 percent by weight of the dispersion.

11. The composition of claim 10, wherein water is present from about 9 to about 10 percent by weight of the dispersion.

12. The composition of claim 1, wherein soap pellets are present from about 90 to about 95 percent by weight of the soap product composition.

13. The composition of claim 12, wherein soap pellets are present from about 93 to about 95 percent by weight of the soap product composition.
